(19) United States
(12) Patent Application Publication
(10) Pub. No.: US 2012/0220892 A1
(43) Pub. Date: Aug. 30, 2012

(54) VIBROMETERS, VIBROMETRIC SYSTEMS, AND METHODS FOR MEASURING SENSORY THRESHOLD

(75) Inventors: Minu Shikha Gandhi, Houston, TX (US); Stacy J. Morris Bamberg, Salt Lake City, UT (US); Christian Brandt Redd, Provo, UT (US); Richard F. Sesek, Auburn, AL (US); Robert P. Tuckett, Sandy, UT (US)

(73) Assignee: THE UNIVERSITY OF UTAH, Salt Lake City, UT (US)

(21) Appl. No.: 13/404,769

(22) Filed: Feb. 24, 2012

Related U.S. Application Data
(60) Provisional application No. 61/446,591, filed on Feb. 25, 2011.
(60) Provisional application No. 61/475,564, filed on Apr. 14, 2011.

(51) Int. Cl. A61B 5/00

(52) U.S. Cl. 600/552

(57) ABSTRACT

Embodiments of the present disclosure relate to vibrometers, vibrometric systems, and methods of using the same. In particular, some embodiments of the vibrometers are capable of producing vibrotactile stimulation at any frequency between 1 and 500 Hz. Moreover, some embodiments of the vibrometer and/or vibrometric system are capable of applying vibrotactile stimulation to a test subject's skin to determine the existence of peripheral neuropathy. Additionally, some embodiments of the method can provide test subjects with comparative vibrotactile threshold test results that can aid in discovering and/or managing of peripheral neuropathy.

### CROSS-REFERENCE TO RELATED APPLICATIONS

The present application claims the benefit or priority to U.S. Provisional Patent Application No. 61/446,591, entitled “Enhanced Vibrometer for Measurement of Sensory Threshold,” filed on Feb. 25, 2011 and U.S. Provisional Patent Application No. 61/475,564, entitled “Enhanced Vibrometer for Measurement of Sensory Threshold and Methods of Operating Same,” filed on Apr. 14, 2011, the entireties of which are incorporated by this reference.

### BACKGROUND OF THE INVENTION

1. The Field of the Invention

This invention relates to systems, methods, and apparatus for measuring sensory threshold.

2. Background and Relevant Art

Peripheral neuropathy, generally, is damage to nerves of the peripheral nervous system. Various forms of peripheral neuropathy can present themselves as seriously debilitating conditions, which can adversely affect quality of life as well as work performance. For example, peripheral neuropathy can result in loss of sensation that can lead to injury from loss of awareness (e.g., burns, cuts, etc.). Additionally or alternatively, peripheral neuropathy can result in “positive” sensation, such as pain.

Neuropathies can have various causes. In some instances, peripheral neuropathy can be caused by chemical exposure such as chemotherapy treatments. Peripheral neuropathy can also result from metabolic disorders, such as diabetes. Furthermore, peripheral neuropathy can result from mechanical compression of a nerve, such as carpal tunnel syndrome (CTS). Although CTS can occur for various reasons (e.g., obesity, oral contraceptives, hyperthyroidism, arthritis, etc.), repetitive motion is a common cause of CTS. Accordingly, in some instances, peripheral neuropathy can be prevented or ameliorated in its extent by accurate and timely diagnosis.

Typically, peripheral neuropathy can be diagnosed with electrophysiological instruments (e.g., instruments used to perform nerve conduction velocity studies), which apply suprathreshold electrical voltage to peripheral nerves. Such diagnosis, however, oftentimes cannot measure unmyelinated neuron injury, because the stimulated current densities necessary to activate unmyelinated fibers can cause tissue injury. Also, to perform the diagnosis using typical electrophysiological instruments requires a trained specialist, who can conduct the study and analyze its results.

Vibrotactile threshold (VT) evaluation also has been used for detecting peripheral neuropathy. However, common VT instrumentation lacks the capability of providing adequate stimulation at low frequencies (e.g., below 30 Hz or below 4 Hz) which may be needed to effectively measure damage to small nerve fibers, including unmyelinated or C-fibers. In particular, VT instrumentation that is incapable of providing required stimulation at low frequencies cannot activate certain mechanoreceptors, such as slowly adapting type I mechanoreceptors. Hence, typical VT instruments cannot accurately determine the existence of and/or completely quantify certain peripheral neuropathies. Damage to such nerve fibers can lead to loss of protective sensation due to decreased function of pain-signaling neurons and loss of blood flow regulation of the skin. Without treatment, such damage can eventually lead to skin ulcerations, which can lead to amputation of a hand, foot, or other body part, and potentially to loss of life

Accordingly, a need exists for systems, methods, and apparatus for measuring sensory threshold.

### BRIEF SUMMARY

Embodiments of the present disclosure provide systems, methods, and apparatus for diagnosing peripheral neuropathy. In at least one embodiment, a vibrometer (or a vibrometric system incorporating the vibrometer) can provide a test subject with vibrotactile stimulation necessary to activate mechanoreceptors on the test subject's skin. In particular, the vibrometer can activate a desired group of mechanoreceptors by stimulating the test subject's skin at a predetermined frequency or range of frequencies and/or at a predetermined displacement (i.e., amplitude) or range of displacements. Additionally, the vibrometric system can apply vibrotactile stimulus at various locations on the test subject skin as well as in various, preset positions or ranges of positions of the test subject's extremities, which can aid in determining existence of peripheral neuropathy. Although described in conjunction with the hand as the test subject's extremity, other extremities, such as the feet and metatarsals, and/or other body parts may be examined.

In one or more embodiments, a device for measuring vibrotactile threshold can include a housing, an actuator coupled to the housing, and a probe functionally connected to the actuator. In some embodiments, the device can be configured to produce displacement of the probe at a predetermined distance or range of distances. In other embodiments, the device can be configured such that the displacement of the probe can occur at a frequency or range of frequencies. In further embodiments, the device can be configured to both produce displacement of the probe at a predetermined distance or range of distances and that displacement of the probe can occur at a frequency or range of frequencies.

Moreover, displacement of the probe, while in contact with the test subject's skin, can produce vibrotactile stimulation, which can activate certain mechanoreceptors and/or groups thereof. More specifically, certain mechanoreceptors and/or groups of mechanoreceptors can be activated at particular frequencies and/or displacements of the probe. Furthermore, as described below in more detail, iterative presentation of the vibrotactile stimulation can aid in determining an activation frequency and/or amplitude (i.e., displacement of the probe) of certain mechanoreceptors and groups of mechanoreceptors). Additionally, the displacement of the probe can be increased at lower frequencies, which can enhance or increase activation of mechanoreceptors and/or groups of mechanoreceptors.

Hence, in some embodiments, the vibrometer can generate vibrotactile stimulation (produced by movement of the probe) at very low frequencies, which can range from 0.1 Hz to about 10 Hz. Additionally or alternatively, the vibrometer also can operate at medium frequencies, which can range from about 8 Hz to 100 Hz. The vibrometer also can operate at high frequencies, which can range from about 90 Hz to about 500 Hz. Moreover, the vibrometer can operate at frequencies above 500 Hz, for example, in the range of 400 Hz to 20,000 Hz. In at least one embodiment, the vibrometer can operate at any frequency in the range from approximately 0.1 Hz to 20,000 Hz.

As described above, displacement of the probe also can vary with the frequency thereof. In particular, frequencies in hundreds or thousands of Hz range can activate mechanoreceptors at a relatively small amplitude (i.e., at a small displacement of the probe). For example, the vibrometer can produce vibrotactile stimulation at about 500 Hz and at an amplitude of 1 μm (i.e., the displacement of the probe can be 1 μm), which can activate certain mechanoreceptors. By contrast, at low frequencies, the activation amplitude can be greater than at higher frequencies. For instance, at frequencies between 0.1 and 4 Hz, the activation amplitude (i.e., the displacement of the probe that activate certain mechanoreceptors) can be in the range of 100 μm to 3,000 μm. It should be noted, that the activation amplitude at a particular frequency can vary from one test subject to another test subject as well as from one location to another location on the same test subject's skin.

In one or more embodiments, a device for measuring vibrotactile threshold can include a housing, an actuator coupled to the housing, and a probe functionally connected to the actuator. The actuator can be configured to produce movement of the probe between 1 and 1500 μm. Furthermore, the actuator and the probe can be configured such that the movement of the probe can occur at any frequency between 1 and 500 Hz. The device also can include a surround secured to the housing and at least partially encompassing the probe.

At least one embodiment includes a vibrometric system for measuring vibrotactile threshold for determining existence of peripheral neuropathy in a patient. The vibrometric system can have a housing and a vibrometer having a moveable probe and an actuator functionally coupled to the movable probe. The probe can be configured to move at any frequency between 1 and 500 Hz. The vibrometer can be rotatably coupled to the housing. Additionally, the system can include a controller configured to direct the actuator to move the probe of the vibrometer at a preset frequency and displacement. The controller also can be configured to receive input from a test subject.

Additionally, at least one embodiment includes a method of determining the existence of peripheral neuropathy in/on a patient's skin. The method can include presenting a test subject with a first time period during which vibrotactile stimulation is applied to a test subject's skin to stimulate a desired group of mechanoreceptors. Also, the method can include receiving a first feedback from the test subject related to the test subject's sensation during the first time period. Moreover, the method can include presenting the test subject with a second time period during which vibrotactile stimulation is not applied to the test subject's skin. The method also can include receiving a second feedback from the test subject related to the test subject's sensation during the second time period. Furthermore, the method can include comparing the second feedback received from the test subject related to the test subject's sensation during the second time period and the first feedback received from the test subject related to the test subject's sensation during the first time period to design values.

Additional features and advantages of exemplary embodiments of the invention will be set forth in the description which follows, and in part will be obvious from the description, or may be learned by the practice of such exemplary embodiments. The features and advantages of such embodiments may be realized and obtained by means of the instruments and combinations particularly pointed out in the appended claims. These and other features will become more fully apparent from the following description and appended claims, or may be learned by the practice of such exemplary embodiments as set forth hereinafter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. For better understanding, the like elements have been designated by like reference numbers throughout the various accompanying figures. Understanding that these drawings depict only typical embodiments of the invention and are not therefore to be considered to be limiting of its scope, the invention will be described and explained with additional specificity and detail through the use of the accompanying drawings in which:

FIG. 1 illustrates a perspective view of a vibrotactile testing fixture in accordance with one embodiment of the present disclosure;

FIG. 2A illustrates a perspective view of a vibrometer in accordance with one embodiment of the present disclosure;

FIG. 2B illustrates an exploded view of the vibrometer of FIG. 2;

FIG. 3 illustrates a top view of a flexure of a vibrometer in accordance with one embodiment of the present disclosure;

FIG. 4 illustrates a flow chart of a vibrotactile testing procedure in accordance with one embodiment of the present disclosure;

FIG. 5A illustrates a flow chart of another vibrotactile testing procedure in accordance with one embodiment of the present disclosure;

FIG. 5B illustrates a flow chart of yet another vibrotactile testing procedure in accordance with one embodiment of the present disclosure;

FIG. 6 illustrates a flow chart of another aspect of vibrotactile testing procedure in accordance with one embodiment of the present disclosure; and

FIG. 7 illustrates a flow chart of yet another aspect of vibrotactile testing procedure in accordance with one embodiment of the present disclosure.

### DETAILED DESCRIPTION OF THE PREFERRED EMBODIMENTS

Embodiments of the present disclosure provide systems, methods, and apparatus for diagnosing peripheral neuropathy. In at least one embodiment, a vibrometer (or a vibrometric system incorporating the vibrometer) can provide a test subject with vibrotactile stimulation necessary to activate mechanoreceptors on the test subject's skin. In particular, the vibrometer can activate a desired group of mechanoreceptors by stimulating the test subject's skin at a predetermined frequency. Additionally, the vibrometric system can apply vibrotactile stimulus at various locations on the test subject skin as well as in various, preset positions or ranges of positions of the test subject's extremities or other body parts, which can aid in determining existence of peripheral neuropathy.

In one or more embodiments, the vibrometric system can include a vibrotactile threshold testing fixture, which can include a vibrometer that can apply vibrotactile stimulation to a test subject's skin. The vibrometric system also can include a controller for regulating the vibrotactile stimulation. For instance, the controller can regulate the frequency of the vibrotactile stimulation generated by the vibrometer. Additionally or alternatively, the controller can regulate the displacement of the probe (i.e., the amplitude of the vibrotactile stimulation generated by the vibrometer).

Embodiments of the present invention may comprise or utilize a special purpose or general-purpose computer including computer hardware, such as, for example, one or more processors and system memory, as discussed in greater detail below. Embodiments within the scope of the present invention also include physical and other computer-readable media for carrying or storing computer-executable instructions and/or data structures. Such computer-readable media can be any available media that can be accessed by a general purpose or special purpose computer system. Computer-readable media that store computer-executable instructions in the form of data are computer storage media. Computer-readable media that carry computer-executable instructions are transmission media. Thus, by way of example, and not limitation, embodiments of the invention can comprise at least two distinctly different kinds of computer-readable media: computer storage media and transmission media.

Computer storage media includes RAM, ROM, EEPROM, CD-ROM, solid state drives (SSDs) that are based on RAM, Flash memory, phase-change memory (PCM), or other types of memory, or other optical disk storage, magnetic disk storage or other magnetic storage devices, or any other medium which can be used to store desired program code means in the form of computer-executable instructions, data or data structures and which can be accessed by a general purpose or special purpose computer.

Further, upon reaching various computer system components, program code means in the form of computer-executable instructions or data structures can be transferred automatically from transmission media to computer storage media (or vice versa). For example, computer-executable instructions or data structures received over a network or data link can be buffered in RAM within a network interface module (e.g., a network interface card or “NIC”), and then eventually transferred to computer system RAM and/or to less volatile computer storage media at a computer system. Thus, it should be understood that computer storage media can be included in computer system components that also (or even primarily) utilize transmission media.

Computer-executable (or computer-interpretable) instructions comprise, for example, instructions that cause a general purpose computer, special purpose computer, or special purpose processing device to perform a certain function or group of functions. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

The vibrometer can incorporate a probe that can contact the test subject's skin and provide the vibrotactile stimulation thereto. Accordingly, the controller can regulate the frequency and/or displacement of the probe's movement. The controller also can receive input from the test subject. For example, the controller can receive feedback related to vibrotactile stimulation from the test subject—e.g., input related to sensation experienced by the test subject. Such feedback from the test subject also can be related to vibrotactile stimulation provided during a predetermined period of time.

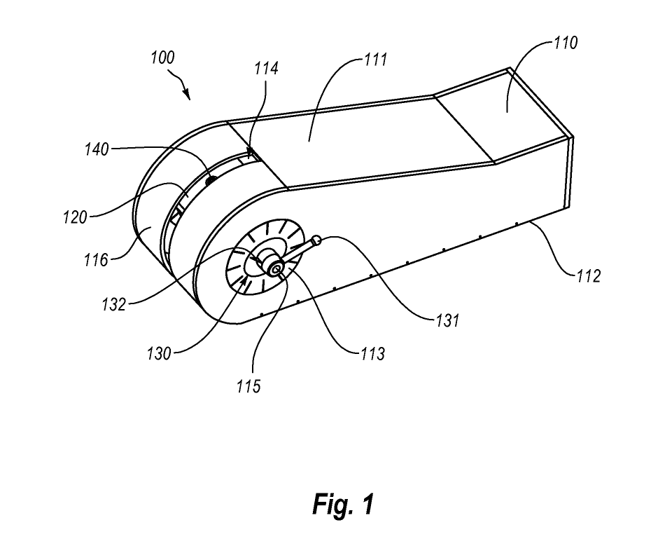

As described above, the vibrometric system can include a vibrotactile threshold testing fixture and a controller. In particular, as illustrated in FIG. 1, vibrotactile threshold (VT) testing fixture 100 can have a housing 110 that secures one or more vibrometers 120. For instance, the vibrometer 120 can be disposed within the housing 110. Furthermore, in addition to supporting the vibrometers 120, the housing 110 can support, position, and/or secure the test subject's extremity for administering the VT testing.

Hence, in at least one embodiment, a technician can at least partially secure or position the test subject's extremity to or relative to the housing 110 or a part thereof For example, the housing 110 can have an angled top surface 111 that can support, position, and/or secure the test subject's extremity. The top surface 111 also can be substantially flat or can have a curved shape to accommodate a particular extremity of the test subject. Accordingly, for instance, the test subject (with or without assistance of the technician) can place an arm on the angled surface, such that the test subject's arm remains substantially stationary. In some embodiments, the test subject's extremity may be substantially immobilized by, for example, securing the extremity to the housing with a strap or other restraining mechanism.

In one or more embodiments, the test subject's extremity that can be tested is a hand or one or more phalanges of the hand. Hence, the housing 110 can accommodate, position, and/or secure the test subject's hand and/or one or more phalanges. More specifically, the housing 110 can accommodate the test subject's hand at various inflection angles of the test subject's wrist. Moreover, the top surface 111 of the housing 110 can be adjustable, such that it can position and/or secure the test subject's hand at various inflection angles of the test subject's wrist.

For example, the test subject's hand can be positioned in a relaxed or natural inflection of the wrist (i.e., generally aligned with the forearm) for one or more VT testing procedures. Additionally or alternatively, the test subject's hand can be positioned at a fully-bent inflection (upward or downward) of the wrist for one or more VT testing procedures. Moreover, the top surface 111 of the housing 110 can accommodate, position, and/or secure the test subject's hand at other intermediate (between the fully-bent inflection upward and fully-bent inflection downward) inflections of the wrist.

Similarly, the vibrometer 120 can be adjusted (e.g., rotated) to accommodate the desired position of the test subject's extremity. For instance, the vibrometer 120 can be rotated to be in a position where the probe 140 can contact the subject's hand or phalange when the hand is in the natural inflection position of the wrist. The vibrometer 120 also can be rotated and/or adjusted to positions where the probe 140 can contact a desired portion of the hand, such as at various inflections of the wrist (described above).

Furthermore, the housing 110 can include inserts, which may be removable and/or disposable and which can have various desired shapes that can accommodate, position, and/or secure the test subject's extremity in desired positions (e.g., as described above). For example, the top surface 111 can incorporate such insert. The insert can be shaped to better (or more ergonomically) accommodate the test subject's extremity receiving the vibrotactile stimulation. Additionally, the insert can be shaped to orient the test subject's extremity in a desired position. Also, the insert can be shaped to accommodate a specific extremity, such as a leg, a foot, etc. Moreover, the inserts can have various sizes that can closely match the size of the test subject's extremity receiving the vibrotactile stimulation.

The housing 110 also can have a bottom surface 112, which can support and orient the housing 110 on a support surface. For instance, the technician can set the VT testing fixture 100 on a table or other similar surface to perform VT tests on the test subject's skin. Accordingly, the bottom surface 111 of the VT testing fixture 100, which may be horizontal, can be in contact with the support surface and can provide orientation and stability to the VT testing fixture 100. Surfaces 111, 112 can have various planar orientations with respect to each other as well as with respect to the housing 110. Additionally, the surfaces 111, 112 can have various suitable shapes (e.g., as described above) and can be continuous or interrupted; for example, the bottom surface 111 can comprise multiple adjustable feet.

In one or more embodiments, the vibrometer 120 can be rotatably coupled to the housing 110. For example, a rotating assembly 130 can rotatably couple the vibrometer 120 to the housing 110. In particular, the rotating assembly 130 can include a handle 131 and a locking mechanism 132, which can secure the vibrometer 120 at a desired angle. Thus, the technician can administer VT testing at different positions of the test subject's extremity (e.g., at a bent and unbent positions, at multiple locations on the extremity, or a combination thereof).

The vibrometer 120 can be manually rotated or can be automated and directed to rotate by the controller (not shown). It should be appreciated that various manual and/or automated mechanisms can be used to rotate the vibrometer 120. In the illustrated example of FIG. 1, the technician can rotate the vibrometer 120 using the handle 131, thereby positioning the vibrometer 120 at a desired angle. The vibrometer 120 can be subsequently secured by the locking mechanism 132, which, for example, can include a nut that can apply pressure onto an enclosure of the vibrometer 120, thereby securing the vibrometer 120 at the desired angle. In other embodiments, the rotating assembly may include a locking feature, such as a ratchet or other locking mechanism, that facilitates positioning of the test subject at desired increments.

The VT testing fixture 100 also can incorporate an angle readout 113 that can have a visible scale in radians, degrees, or other identifiable metrics. Such angle readout 113 can be mounted on a side of the housing 110. Alternatively, the angle readout 113 can comprise a rotary encoder or other position sensor that can communicate with the controller or a display to provide rotational information about the vibrometer 120. Hence, the angle readout 113 can be used to set the vibrometer 120 at the desired angular position.

The vibrometer 120 also can incorporate a probe 140 that can make contact with the test subject's extremity that is being examined. In particular, the probe 140 can vibrate at a predetermined frequency and/or displacement, as determined by the controller, to provide vibrotactile stimulation to the test subject's extremity. Thus, when the test subject places its extremity onto the VT testing fixture 100, the probe 140 of the vibrometer 120 can provide vibrotactile stimulation to the extremity or other body part through, for example, the subject's skin.

In at least one embodiment, the housing 110 of the VT testing fixture 100 can at least partially shield the probe 140 and/or the vibrometer 120. Hence, the housing 110 can include an opening 114 that can accommodate the probe 140, such that the probe 140 can come into contact with the test subject's skin, but at least a portion of the vibrometer 120 may be concealed and/or shielded within the housing 110. Furthermore, the opening 114 can accommodate rotation of the probe 140 as the vibrometer 120 is rotated about an axis 115.

For example, the housing 110 can have a side surface 116, which can at least partially conceal and/or shield the vibrometer 120. The side surface 116 can incorporate the opening 114, which can allow the probe 140 to protrude past the side surface 116. In one or more embodiments, the side surface 116 can have an arcuate shape. For instance, the side surface 116 can have a semi-cylindrical shape centered on the axis 115 of rotation of the vibrometer 120. Accordingly, as the vibrometer 120 rotates about the axis 115, the probe 140 can uniformly protrude above the side surface 116.

As described above, the probe 140 can protrude above the housing 110, such that the vibrometer 120 can deliver the vibrotactile stimulation to the test subject. The vibrotactile stimulation can be achieved by oscillating movement of the probe 140. For example, the probe 140 can oscillate in a direction substantially perpendicular to a portion of the test subject's skin receiving the vibrotactile stimulation. Essentially, the probe 140 can tap or compress the test subject's skin, to activate the desired mechanoreceptors.

Alternatively, the direction of the oscillatory movement of the probe 140 can be other than perpendicular to the portion of the test subject's skin receiving the vibrotactile stimulation. Generally, however, the oscillatory movement of the probe 140 can be in such direction that in response to the movement of the probe 140 the skin of the test subject is compressed. Although some skin stretch may be present when the probe 140 oscillates in a direction other than perpendicular to the portion of the test subject's skin receiving the vibrotactile stimulation, the orientation of the oscillations of the probe 140 can minimize such stretch.

Accordingly, the probe 140 can be recessed below a surface (e.g., below a surround that encompassed the probe 140) upon which the skin receiving the vibrotactile stimulation can rest. Alternatively, the probe 140 can be substantially flush with the surface upon which the skin receiving the stimulation feedback rests. Moreover, the probe 140 also can protrude past the surface upon which the skin receiving the vibrotactile stimulation rests.

Hence, in some instances, the test subject's skin can recess below the surface of initial contact with the skin and can contact the probe 140. Otherwise, the test subject's skin receiving the vibrotactile stimulation can rest on top and/or can be indented or compressed by the probe 140, in a neutral or stationary position of the probe 140 (i.e., prior to commencement of oscillating movement). Furthermore, in at least one embodiment, the skin of the test subject may not be in contact with the probe 140, when the probe 140 is in the neutral or stationary position. Thus, in addition to compressing the test subject's skin, the probe 140 can deliver a predetermined impact thereon.

As described above, the probe 140 of the vibrometer 120 can move at a desired frequencies and amplitudes (displacements of the probe), as regulated by the controller. In at least one embodiment, a linear or a rotary actuator can couple to the probe 140 and can generate movement thereof, as regulated by the controller. Moreover, the controller can direct the actuator to produce movement at any frequency between 1 Hz and 500 Hz. In other words, oscillatory movement of the probe 140 can have limitless number of frequencies between 1 Hz and 500 Hz.

Setting and adjusting frequencies can vary depending on the type of actuator used in the vibrometer 120. For example, a voice coil actuator can receive alternating current, the frequency of which can be set and/or adjusted by the controller. The frequency of a piezoelectric actuator, and consequently of the probe 140, also can be adjusted by adjusting the frequency of alternating current. Alternatively, frequency of the probe 140 of the vibrometer 120 that incorporates a stepper motor (e.g., integrated with a rotary to linear motion converter) as an actuator, can be adjusted by, for example, regulating the rotational speed of the stepper motor. Therefore, the frequency of the actuator and, consequently, of the probe 140 can be continuously adjusted and can be set at desired magnitude.

Similarly, the displacement of the probe 140 can be continuously varied. In particular, the amplitude of the oscillatory movement of the probe 140 can vary between 1 μm and 1500 μm. Furthermore, the controller can increase the amplitude of the oscillations of the probe 140 as the frequency is decreased. For example, at the frequency of 50 Hz, the probe 140 can have 10 μm displacement, and at the frequency of 4 Hz, the probe 140 can have 250 μm displacement.

Furthermore, the displacement of the probe 140 can vary with respect to time as a sine or a triangle wave function. For instance, the alternating current can have a sine or triangle waveform and can move the actuator, such as a voice coil or a piezoelectric actuator, in the sine or triangle waveform oscillations. Alternatively, the controller can regulate the speed of the servo motor to achieve a desired waveform of the oscillations of the probe 140 (e.g., at constant speed, the probe 140 can have triangle waveform oscillations).

Oscillations of the actuator and/or the probe 140, in some instances, can produce audible noise. Accordingly, the test subject can be alerted by such noise, which may lead or contribute to less accurate results of the VT testing procedure. Hence, in one or more embodiments, the VT testing fixture 100 and/or the vibrometric system can include a noise generator, which can mask the noise produced by the actuator and/or by the probe 140. In other embodiments, the test subject may be otherwise shielded from the noise. For example, the test subject may wear earplugs or other noise dampening devices. It may be desirable to isolate the rest of the test subject's body from these vibrations, which may translate through other portions of the housing 110. Thus dampening or other isolating features may limit the amount of vibration experienced by the parts of the body that are not currently being examined.

In some embodiments, such as embodiments where the actuator uses a voice coil, the actuator and/or the probe 140 may generate heat, which, in some instances, can become noticeable, uncomfortable, and/or painful for the test subject. Hence, the vibrometric system (and/or the VT testing fixture 100) may include a cooling system that can cool the actuator, the vibrometer 120, and/or the probe 140. Moreover, the cooling system also can generate noise that can mask the noise generated by the actuator and/or by the probe 140 during oscillations. Accordingly, in at least one embodiment, the cooling system also can function as a noise generator for masking the noise produced in the vibrometric system during the oscillations of the probe 140. In other embodiments, a cooling system may not be necessary. For example, where the actuator is a piezoelectric actuator, the actuator and/or probe may generate minimal heat.

Additionally, the vibrometric system can include one or more temperature sensors that can determine the temperature of the air and/or temperature of the test subject's skin in contact with a specific portion of the VT testing fixture 100. For instance, the VT testing fixture 100 can incorporate temperature sensors (e.g., in the housing 110 of the VT testing fixture 100), which can detect the temperature of the test subject's skin that's in contact with the temperature sensor(s). Additionally or alternatively, the vibrometer 120 can incorporate one or more temperature sensors into, for example, the probe 140, surround, or other portion of the vibrometer 120 or housing 110), which can detect the temperature of the portion of the skin that is in contact with the probe 140 and/or the surround.

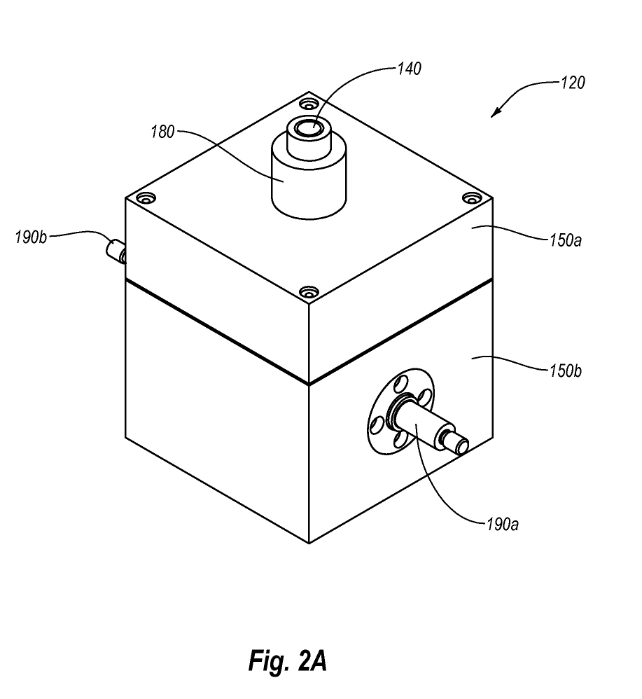

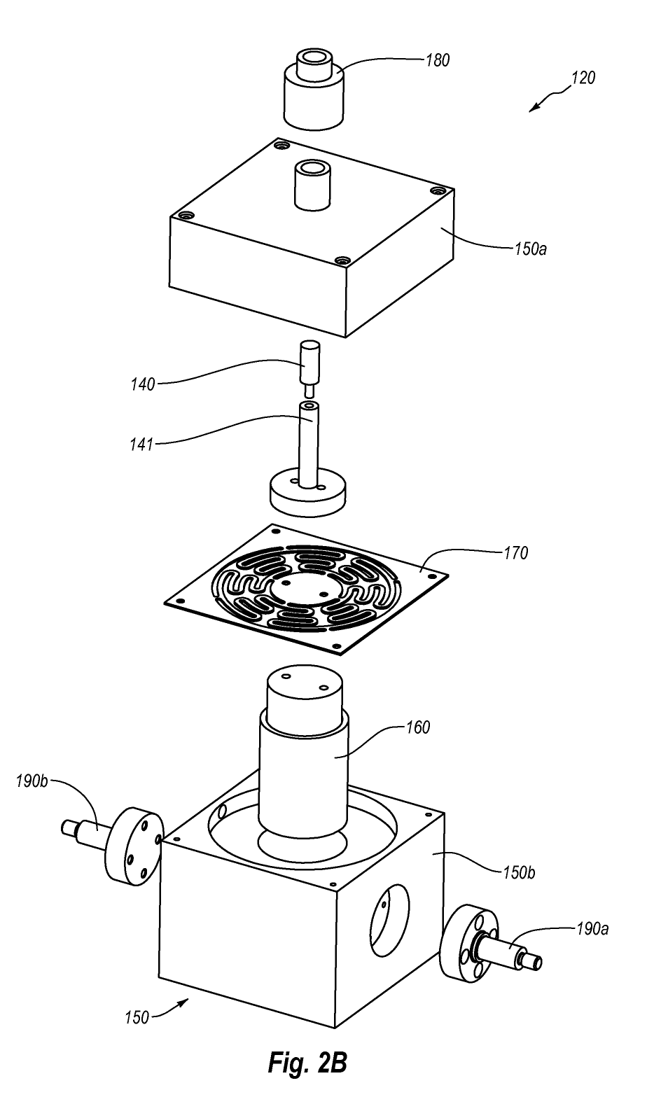

As described above, the probe 140 can come into contact with the test subject's skin to deliver vibrotactile stimulation. Generally, as illustrated in FIGS. 2A and 2B, the probe 140 may be secured and/or housed within an upper enclosure portion 150a and/or within a lower enclosure portion 150b. Furthermore, the upper and lower enclosure portions 150a, 150b can house, secure, and/or conceal various elements and/or components of the vibrometer 120.

As shown in FIGS. 2A and 2B, the actuator 160 can be a linear actuator such as a voice coil (moving coil or moving magnet). In other embodiments, other types of actuators 160 may be used. For example, a linear motor, a piezoelectric actuator, a hydraulic or pneumatic cylinder, or other type of linear actuator may be used. Alternatively, the actuator 160 can be a rotary actuator (e.g., a servo motor) or a combination of a rotary actuator and a rotary to linear movement conversion mechanism. The controller can determine and/or regulate the amount of and can supply the power to the actuator 160. For instance, the controller can regulate the frequency and amplitude produced by the actuator 160 by regulating the frequency and voltage supplied to the actuator 160.

In some embodiments, the actuator 160 can couple directly to the probe 140. Alternatively, the probe 140 can be secured to a probe connector 141 (shown in FIG. 2B), which can couple to the actuator 160, and which can serve (for example) as a functional connection between the actuator 160 and the probe 140. Thus, the probe 140 can be removable and/or disposable. Similarly, the probe 140 can be removed, cleaned and/or sterilized, and reconnected to the probe connector 141.

The probe 140 can have various shapes. For instance, the probe 140 can have a substantially cylindrical shape. Additionally, a tip of the probe 140, which contacts the test subject's skin, can have various shapes. For example, the tip of the probe 140 can be substantially flat, spherical, or can have any other desired shape. Similarly, body of the probe 140 can have various suitable shapes, in addition to the above-described cylindrical shape (e.g., rectangular prismoid, triangular prismoid, etc.).

The probe 140 also can have various sizes (lengths, widths, heights, and diameters, as appropriate). In particular, the probe 140 can have a diameter between 1-6 mm. Additionally or alternatively, the probe 140 can have a diameter between 2-10 mm, and can have a diameter greater than 10 mm. The probe 140 also can have a height (i.e., protruding portion) of approximately between 0-2 mm. However, the height of the probe 140 also can be greater than 2 mm.

The actuator 160 can be secured in the upper and/or lower enclosure portions 150a, 150b. For instance, a nonmoving portion of the actuator 160 can be secured in the lower enclosure portion 150b. A moving portion of the actuator 160 can be free to move with respect to the nonmoving portion of the actuator 160 and can be coupled to the probe 140 and/or to the probe connector 141.

Additionally or alternatively, the moving portion of the actuator 160 can be mounted on or suspended from a spring system 170. In particular, the spring system 170 can be secured within the upper and/or lower enclosure portions 150a, 150b such that a portion of the spring system 170 is nonmoving (a stable portion) and another portion of the spring system 170 can move (a flexing portion) in response to the movement of the moving portion of the actuator 160. Similarly, in lieu of or in addition to coupling to the actuator 160, the probe 140 and/or probe connector 141 can couple to the moving portion of the spring system 170. The spring system 170 is generally used in connection with a voice coil type actuator. However, the spring system 170 may be used in conjunction with other actuators 160.

In one or more embodiments, the vibrometer 120 can incorporate a surround 180. The surround 180 can at least partially encompass the probe 140. Surrounding the probe 140 can in some instances improve the reliability of the VT test. In particular, the surround 180 can isolate a portion of the test subject's skin that receives the vibrotactile stimulation, which can aid in isolating particular mechanoreceptors on the skin.

The size of the surround 180 can depend on the size of the probe 140. Generally, the surround 180 can form a gap of approximately 1.5±0.6 mm between an opening in the surround 180 and the external dimensions of the actuator 160. Accordingly, the surround 180 can be, for example, approximately cylindrical and can have internal diameter that is between 2 and 8 mm. As described above, however, the probe 140 also can have a diameter that is greater than 6 mm. Accordingly, the internal diameter of the surround 180 also can be greater than 7.5 mm, to accommodate a correspondingly sized probe 140. Other shapes and sizes are also contemplated.

The surround 180 also can be secured to the upper and/or lower enclosure portions 150a, 150b or can be otherwise secured. Hence, the surround 180 can remain stationary during the movement of the probe 140. Therefore, a test subject's skin contacting the surround 180 can isolate the skin that is in contact with the probe 140, which can improve the VT test's accuracy.

Additionally, the vibrometric testing system can measure the force exerted by the test subject onto the surround 180. For example, the surround 180 can incorporate a force sensor, which can determine the amount of force exerted by the test subject onto the surround 180. The force sensor can send the force information to the controller, and this information can be used in evaluating the accuracy of the test results.

The vibrometer 120 also can include one or more axles (or axle shafts) 190 (e.g., axles 190a and 190b). The axles 190 can support and/or secure the vibrometer 120 within the housing 110. Additionally, the axles 190 can provide the vibrometer 120 with freedom to rotate about one or more axes—e.g., when there is a single axle 190 or multiple axles 190 that align along a single axis passing through center(s) of the axle(s), the vibrometer 120 may rotate about the one or more axes that pass through the center of the axles 190. One or more of the axles 190 can be aligned substantially with a center of the upper and/or lower enclosure portions 150a, 150b, a center axis of the probe 140, and/or a center axis of the actuator 160. Alternatively, axles 190 can have other alignments.

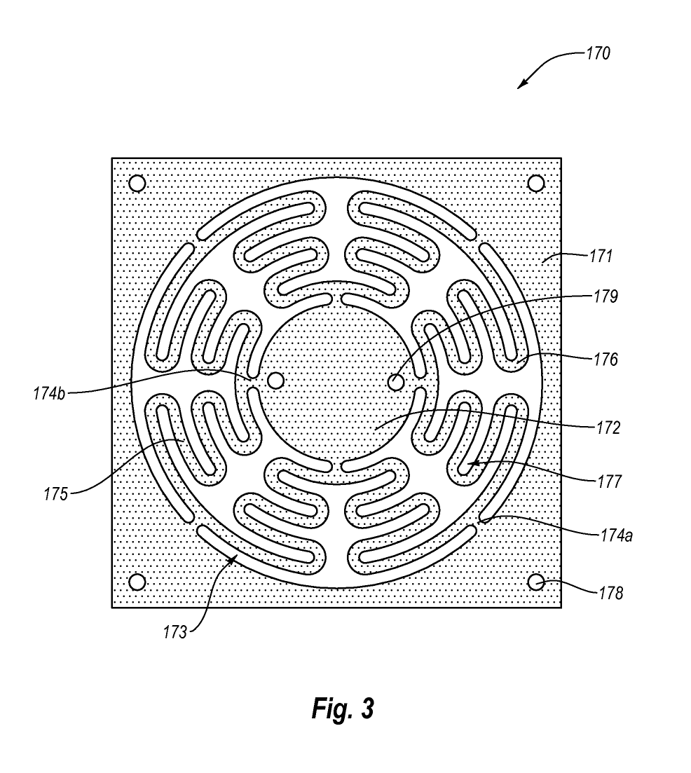

As described above, the vibrometer 120, in some embodiments, can include a spring system 170. For instance, the spring system 170 can comprise a flexure, as illustrated in FIG. 3. The flexure can be made from essentially any spring-like material. For example, the flexure can be made from a sheet of spring steel. Additionally, the flexure made from spring steel may be heat treated to obtain particular qualities of the spring steel.

The flexure may include a stable portion 171 and a flexing portion 172. The flexing portion 172 can be connected to the stable portion 171 with a plurality of struts 173. Such struts 173 can couple to the stable portion 171 at connection points 174a. Similarly, the struts 173 can couple to the flexing portion 172 at connection points 174b. In the embodiment, illustrated in FIG. 3, the struts 173, collectively, have four connection points 174a and four connection points 174b. Additionally, in the embodiment, the flexure incorporates a total of eight struts. Hence, at least two struts 173 share at least one connection point 174a and at least two struts 173 share at least one connection point 174b.

Alternatively, each strut 173 can have one or more connection points 174a, 174b, connecting the strut 173 to the stable portion 171 and/or to the flexing portion 172. Moreover, two or more struts 173 can share multiple connection points 174a and/or connection points 174b. Accordingly, the struts 173 can be designed to provide sufficient rigidity to the spring system 170.

In one or more embodiments, the struts 173 can comprise one or more ribbons 175. For example, a single ribbon can be used to form all of the struts 173. Alternatively, multiple ribbons 175 can be used to make the struts 173 (e.g., one ribbon 175 can be used to form one strut 173). Moreover, the ribbon 175 can have a plurality of transition points 176, which form loops 177.

Such loops 177 can include portions of the ribbon 175 that can be parallel one to another. Additionally or alternatively, loops 177 also can include non-parallel portions of the ribbon 175. For instance, loops 177 can have non-parallel portions of the ribbon 175 that are disposed at a predetermined angle with respect to each other. Furthermore, the portions of the ribbon 175 that form loops 177 can have a substantially linear shape. Alternatively, however, the portions of the ribbon 175 forming loops 177 can have arcuate, irregular, or other desired shape. Similarly, loops 177 forming one strut 173 can be different from loops 177 forming another strut 173 of the spring system 170.

As described above, the spring system 170 can be secured within the upper and/or lower enclosure portions 150a, 150b. In at least one embodiment, the spring system 170 can have one or more mounting holes. For example, the stable portion 171 of the spring system 170 can have four mounting holes 178. Alternatively, the spring system 170 can be secured within the upper and/or lower enclosure portions 150a, 150b using other fastening mechanisms, such as adhesives, press fitting, clamping, etc.

Similarly, the flexing portion 172 can secure the actuator 160, probe connector 141, and/or probe 140. Thus, the flexing portion 172 can include mounting holes 179, which can be used to secure the actuator 160, probe connector 141, and/or probe 140. In other embodiments, the actuator 160, probe connector 141, and/or probe 140 can be secured the flexing portion 172 of the spring system 170 using other fastening mechanism, as described above in connection with the spring system 170. In further embodiments, the flexing portion 172 may be omitted.

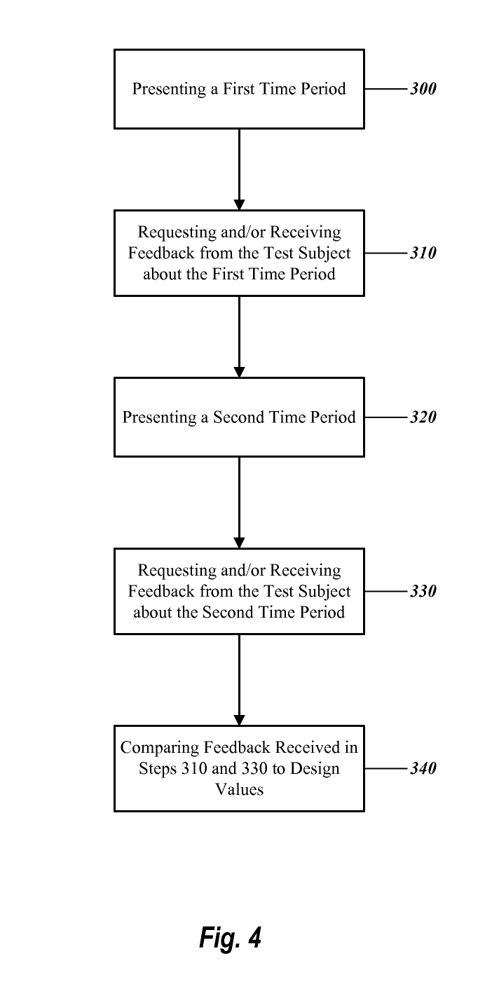

As described above, the probe 140 can contact the test subject's skin to provide vibrotactile stimulation. To administer the VT test, the technician and/or the vibrometric system can instruct the test subject to provide necessary input to the controller. For example, as illustrated in FIG. 4, the test subject can be presented with a first time period (step 300), during which the vibrometric system can present vibrotactile stimulation or can withhold vibrotactile stimulation (presenting still or stationary probe 140). The duration of the time period can be, for instance, from 0.5 seconds to 2 seconds. Alternatively, duration of the time period can be greater than 2 seconds or less than 0.5 seconds.

Furthermore, oscillations of the probe 140 (i.e., vibrotactile stimulation) can be presented to the test subjected at a single or multiple frequencies during the first time period. For example, the test subject can be presented with vibrotactile stimulation at 4 Hz for a time period of 1 second. It should be noted that vibrotactile stimulation applied at the frequency of 4 Hz or below can activate slowly adapting type I mechanoreceptors. The vibrometric system can signal the test subject when the first time period begins and/or when the first time period ends (e.g., with sound, lights, or other stimuli).

After at least partial completion of the first time period, the vibrometric system can request and/or receive feedback from the test subject about the sensations felt by the test subject during the first time period (step 310). For instance, the test subject may have to choose between two options: “felt vibrotactile stimulation” and “did not feel vibrotactile stimulation.” Hence, for example, the test subject can press one of two buttons (actual or virtual) to input the feedback into the controller.

Additionally, the test subject can be presented with a second time period, during which vibrotactile stimulation may be applied to the test subject's skin or the probe 140 may be in a stationary position (step 320). In one or more embodiments, if vibrotactile stimulation was applied during the first time period (step 300), then a still period (i.e., a time period where the probe 140 is stationary) can be presented to the test subject during the second time period (step 320). Additionally or alternatively, presentation of vibrotactile stimulation or a still period can be randomly selected for the first and/or second time periods and/or for additional time periods.

Furthermore, as described above, the frequency of the vibrotactile stimulation presented during the second time period can be the same as the frequency presented during the first time period. In one or more embodiments, the frequency presented during the first time period can be greater than the frequency presented during the second time period. Alternatively, the frequency of the vibrotactile stimulation presented during the first time period can be less than the frequency presented during the second time period. Moreover, the frequency presented during the first and/or the second time periods can vary (increasing and/or decreasing) during the time period.

Similarly, the amplitude of the vibrotactile stimulation presented during the second time period can be the same as the amplitude presented during the first time period. However, the amplitude presented during the first time period also can be greater than the amplitude presented during the second time period. Alternatively, the amplitude of the vibrotactile stimulation presented during the first time period can be less than the amplitude presented during the second time period. Moreover, the amplitude presented during the first and/or the second time periods can vary (increasing and/or decreasing) during the time period.

Similar to the first time period, the duration of the second time period can be from 0.5 seconds to 2 seconds, or can be greater than 2 second or less than 0.5 second. Also, the duration of the second time period can be the same or substantially the same as the duration of the second time period. Alternatively, however, the duration of the second time period can be longer or shorter than the duration of the first time period.

After at least partially presenting the second time period to the test subject (step 320), the vibrometric system can request and/or receive feedback from the test subject about sensation felt by the test subject during the second time period (step 330). The test subject can be signaled or alerted to the end of the second time period in a similar way as described above in connection with the first time period. In step 330, the test subject can provide similar or the same type of feedback as described in connection with step 310.

After the test subject's feedback has been received in steps 310 and/or 330, the vibrometric system can compare the received feedback with design values. In particular, the vibrometric system can analyze whether the test subject correctly determined presence of vibrotactile stimulation. Furthermore, the vibrometric system can compare the results with statistical data for the same demographic of test subjects (e.g., sex, age, gender, build, body-mass index, etc.).

For example, the vibrometric system can consider inaccurate detection of the vibrotactile stimulus as an indication that the test subject cannot feel the vibrotactile stimulus at the frequency and/or amplitude that was set during the first and second time periods. Accordingly, the vibrometric system can compare such indication with an average response of test subjects (e.g., of the same or similar demographic) to the same or similar frequency and/or amplitude of vibrotactile stimulation. In other words, the vibrometric system can compare the indicated condition of the test subject's sensitivity to vibrotactile stimulation with a “normal” sensitivity to similar or same vibrotactile stimulation, in order to determine any deviations from or decrease in the sensitivity of the test subject.

Additionally, the vibrometric system also can include a pause between the first time period and the second time period. Hence, the test subject can input feedback after the first time period has been presented and before the second time period commences. In at least one embodiment, the pause between the time first and second time periods can be approximately 0.5 seconds. Alternatively, the pause can be greater or less than 0.5 seconds.

Furthermore, the vibrometric system may require feedback from the test subject after the first time period has been presented and before commencing the second time period. In other words, the second time period can be presented only after the test subject inputs feedback related to the sensation experienced during the first time period. Accordingly, the test subject can control the duration of the pause between the first and second time periods.

Alternatively, the vibrometric system can require and/or permit entry of feedback after presenting both the first and second time periods. Thus, the test subject would have to input feedback on sensations experienced during the first and second time periods after completion of the steps 300 and 320. Furthermore, the vibrometric system can allow the test subject to input the feedback related to the sensation of the first time period any time during the first time period, during a pause between the time periods (if any), and/or during and after the second time period. Similarly, the vibrometric system can allow the test subject to input feedback any time during or after the second time period.

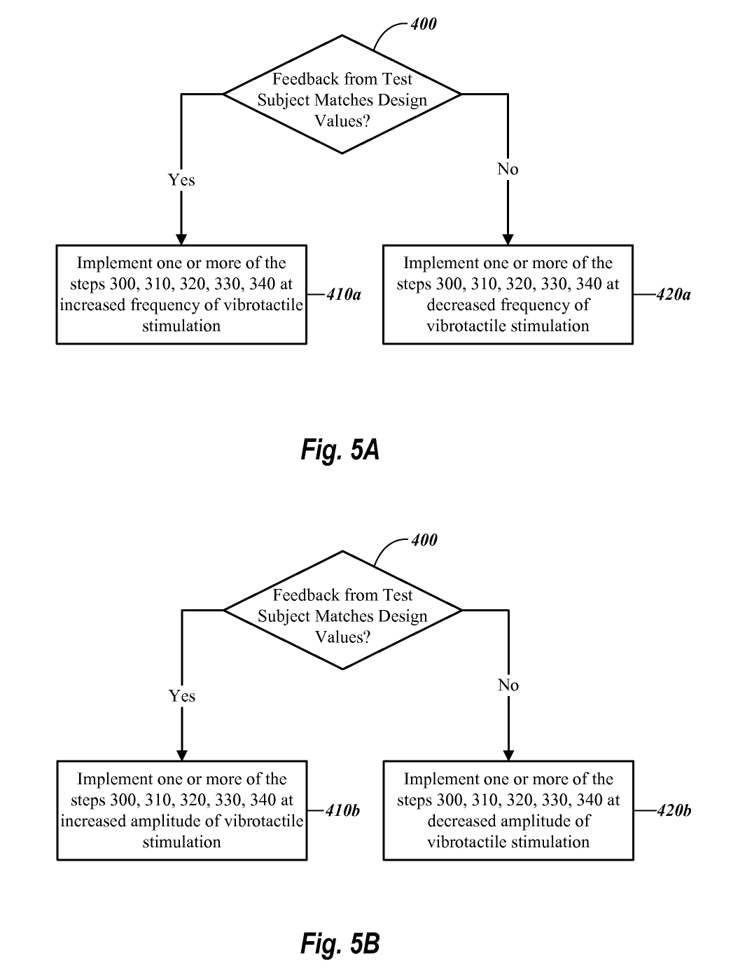

The vibrometric system also can repeat steps 300, 310, 320, 330, and/or 340, while changing the frequency of the vibrotactile stimulation. For instance, if the test subject's feedback is inconsistent with the actual presence or absence of the vibrotactile stimulation during the first and/or the second time periods, the vibrometric system and/or the technician can repeat the steps 300, 310, 320, 330, and/or 340 at the same frequency of frequencies as the original test. Alternatively, as illustrated in FIG. 5A, the vibrometric system and/or the technician can repeat the steps 300, 310, 320, 330, and/or 340 at an increased frequency or frequencies.

More specifically, if the feedback from the test subject does not match one or more of the design values (e.g., feedback is not accurate for both the first and the second time periods), as determined in step 400, one or more of the steps 300 through 340 can be repeated at an increased frequency from the previous test (step 410a). By contrast, if the feedback from the test subject matches the design values (step 400), the frequency of vibrotactile stimulus can be decreased (step 420a). If desired, the test can be repeated iteratively (i.e., after steps 410a and/or 420a, the vibrometric system can commence step 400).

It should be noted that design values can include various types of parameters preset for the VT testing. For instance, the design values can include presence or absence of vibrotactile stimulation during the first and/or second time periods. Accordingly, the feedback received from the test subject about the presence or absence of vibrotactile stimulation during the first and/or the second time periods can be compared with the design values to determine whether the test subject accurately identified the presence or absence of the vibrotactile stimulation. The design values also can include other factors and/or stimuli that may be presented to the test subject during the first and/or second time periods. For example, design values can include presence or absence of variance of frequency or amplitude; presence or absence of heat or cold; duration of stimulation; continuous or interrupted stimulation; direction of stimulation; and other design values.

Moreover, as illustrated in FIG. 5B, if the feedback from the test subject does not match one or more of the design values, as determined in step 400, one or more of the steps 300 through 340 also can be repeated at an increased amplitude from the previous test (step 410b). By contrast, if the feedback from the test subject matches the design values (step 400), the amplitude of vibrotactile stimulus can be decreased (step 420b). If desired, the test can be repeated iteratively (i.e., after steps 410b and/or 420b, the vibrometric system can commence step 400). It should be noted that a test sequence with the same or similar parameters also can be repeated, which may allow the technician and/or test subject to conduct multiple VT tests at the same frequency and/or amplitude. In other words, the test subject can receive the same or similar vibrotactile stimulation during multiple iterations of the first and second time periods, which may improve the accuracy of the indication obtained during the VT testing.

As described above, a particular group of mechanoreceptors can be activated by vibrotactile stimulus at a particular frequency. Hence, increasing and decreasing amplitude of the oscillations (i.e., the displacement) of the probe 140, while maintaining the frequency constant (i.e., the same as in previous VT tests) can help in determining the level of energy required to activate a particular group of mechanoreceptors. Accordingly, ascertaining the amplitude at which a particular group of mechanoreceptors is activated (i.e., activation amplitude), by iteratively repeating steps 400, 410b, and/or 420b, as applicable, can lead to determining and quantifying the presence of peripheral neuropathy.

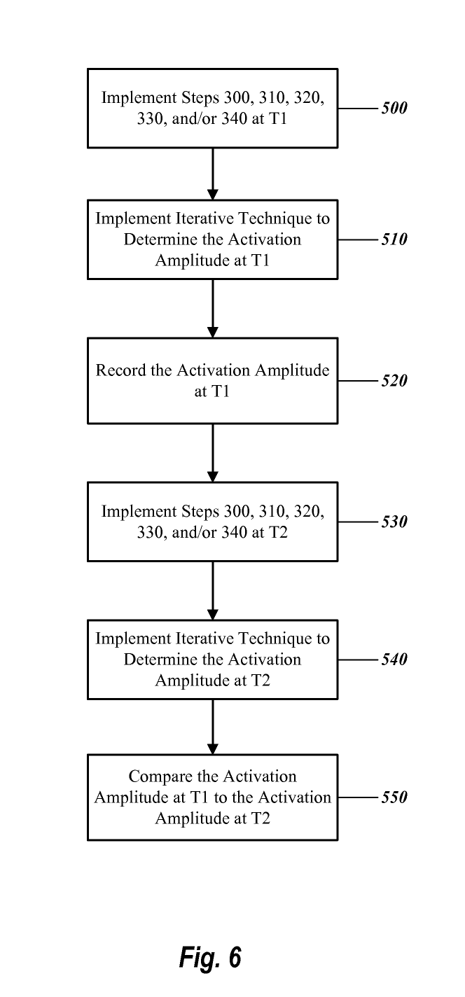

For instance, the determined activation amplitude can be compared to a “normal” activation amplitude, generally exhibited by similarly situated test subjects (i.e., test subjects of at least one of the same demographics, as described above). Alternatively, as illustrated in FIG. 6, the activation amplitude can be tracked through time for the same test subject and results can be compared to determine changes, if any, in the sensory perception of the test subject. Specifically, the VT testing can be performed, using the vibrometric system described herein, on the test subject at a time T1, by implementing one or more of the steps 300, 310, 320, 330, and 340 (step 500). The time T1 can be any date/time desired or chosen by the test subject, vibrometric system, or a third party. Subsequently, one or more activation amplitudes can be determined using the iterative technique, described above, for one or more vibrotactile stimulation frequencies used at the time T1 (step 510), and the activation amplitude for the time T1 can be recorded (step 520).

At a later date/time, at a time T2, the same test subject can undergo VT testing by implementing one or more of the same steps 300 through 340 (step 530). Similarly, one or more activation amplitudes can be determined for one or more frequencies of vibrotactile stimulation used in the VT testing (step 540). Thereafter, in step 550, activation amplitudes at the time T1 can be compared to corresponding activation amplitudes at the time T2 (i.e., to amplitudes for the same frequencies of vibrotactile stimulation).

Comparing the difference or change in the activation amplitude can be particularly useful in determining decrease in tactile sensitivity in the test subject, which can indicate existence and/or progress of peripheral neuropathy. For example, activation amplitude can be determined at time T1 for the test subject that is continuously subjected to one or more of the conditions known to lead to peripheral neuropathy (e.g., repeated motions, chemical exposure, etc.). Subsequent comparison of the activation amplitude at a later time T2 can provide information regarding existence and or progress of peripheral neuropathy in that test subject. Accordingly, the test subject can reduce its exposure (to the extent possible or desirable) to the condition or conditions suspected of contributing to the loss of sensation. Hence, periodic VT testing can help test subjects to eliminate or slow the progress of peripheral neuropathy.

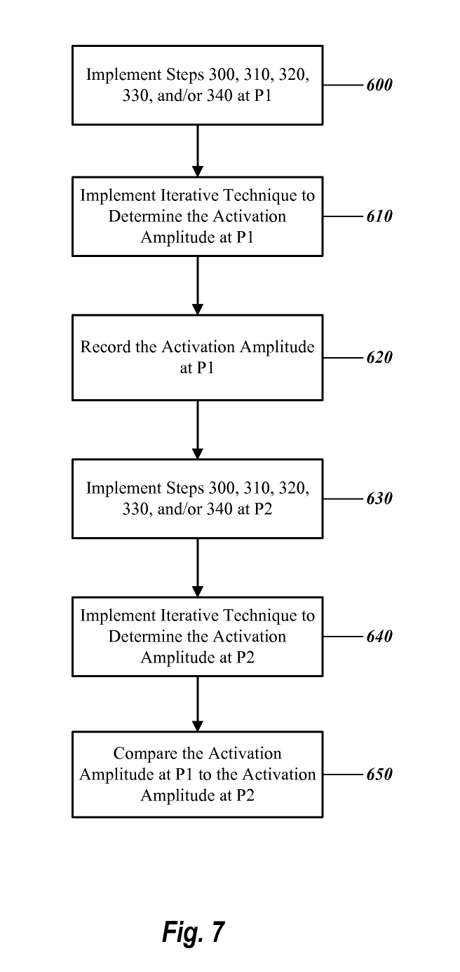

Similarly, as described above, the VT testing can be performed in various positions of the test subject's extremity receiving the vibrotactile stimulation. In particular, as illustrated in FIG. 7, one or more of the steps 300, 310, 320, 330, and/or 340 can be implemented in a first position P1 of the test subject's extremity (step 600). For instance, the first position P1 can be a fingertip on the test subject's hand, while the hand is in a natural (i.e., unbent) inflection of the wrist. Subsequently, the activation amplitude can be determined for the first position P1 (step 610), by implementing the iterative technique described above. In one or more embodiments, the activation amplitude at the first position P1 can be stored (step 620).

Additionally, the test subject's hand can be placed into a second position P2 (e.g., in a fully downward bent inflection of the wrist), and one or more of the steps 300, 310, 320, 330, and/or 340 can be implemented in the second position P2 (step 630). Similarly, the activation amplitude can be determined at the second position P2 (step 640). Thereafter, the activation amplitudes in the first and second positions P1, P2 can be compared (step 650).

Comparing the activation amplitude in the first position P1 to the activation amplitude in the second position P2 can provide information about the existence and/or extent of peripheral neuropathy experienced by the test subject. For example, by comparing the activation amplitude at the test subject's fingertip(s) in the fully downward bent inflection of the wrist to the activation amplitude of the same fingertip(s) in a normal unbent inflection of the wrist, the vibrometric system may determine the presence or absence of carpal tunnel syndrome (CTS). Specifically, an increase in the activation amplitude in the bent inflection of the wrist may indicate presence of CTS.

Furthermore, as alluded to above, the VT testing can be conducted at different temperatures. In one or more embodiments, the VT testing at multiple instances can be conducted at approximately the same room temperature and/or body temperature (e.g., the same body temperature on the portion of the test subject's skin receiving vibrotactile stimulation) at each instance of the VT testing. Accordingly, the vibrometric system and/or the technician can make record and compare corresponding temperatures for each activation amplitude determined during VT testing. Furthermore, skin temperature can be monitored continuously throughout the VT testing, to ensure accurate and/or consistent interpretation of the results.

In at least one embodiment, the test subject also can receive conditioning stimulus or stimuli, prior to, during, or after the VT testing. For instance, a brush (e.g., a camel's hair brush) can be gently rubbed or brushed against the test subject's skin. Such brushing can be proximate to the location on the test subject's skin that receives the vibrotactile stimulation. In particular, conditioning stimuli can be administered approximately 5 cm from the probe 140 of the vibrometer 120.

Furthermore, the activation amplitude can be obtained with and without the conditioning stimuli. For example, separate VT tests can be conducted with and without conditioning stimuli. Subsequently, the activation amplitude without the conditioning stimuli can be compared to the activation amplitude obtained during the VT testing with the conditioning stimuli. Moreover, as described above, the test subject can receive conditioning stimuli after receiving the vibrotactile stimulation and/or after obtaining the activation amplitude. Hence, additional input can be obtained from the test subject, which relates to the sensation experienced by the test subject in response to the conditioning stimuli applied after vibrotactile stimulation.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

We claim:

1. A device for measuring vibrotactile threshold, the device comprising:
a housing;
an actuator coupled to the housing;
a probe functionally connected to the actuator, the actuator configured to produce displacement of the probe between 1 and 1500 μm, the actuator and the probe configured such that the displacement of the probe can occur at any frequency between 1 and 500 Hz; and
a surround secured to the housing and at least partially encompassing the probe.

2. The device of claim 1, wherein the displacement of the probe can occur at frequencies below 31.5 Hz.

3. The device of claim 1, wherein the displacement of the probe can occur at frequencies below 31.5 Hz and above 200 Hz.

4. The device of claim 1, wherein the actuator is a linear actuator.

5. The device of claim 1, further comprising a spring system, wherein a moving portion of the actuator is coupled to a flexing portion of the spring system.

6. The device of claim 5, wherein the spring system comprises a flexure.

7. The device of claim 6, wherein the flexure comprises:
the flexing portion of the spring system;
a stable portion; and
a plurality of struts connecting the flexing portion and the stable portion

8. The device of claim 7, wherein the plurality of struts comprises a single ribbon with a plurality of transition points therein.

9. A vibrometric system for measuring vibrotactile threshold for determining the existence of peripheral neuropathy in a patient, the system comprising:
a housing;
a vibrometer having a moveable probe and an actuator functionally coupled to the movable probe, the probe being configured to move at any frequency between 1 and 500 Hz, the vibrometer being rotatably coupled to the housing; and
a controller configured to direct the actuator to move the probe of the vibrometer at a preset frequency and displacement and is further configured to receive input from a test subject.

10. The vibrometric system of claim 9, wherein the housing is configured to position the test subject's hand at various inflections of the test subject's wrist.

11. The vibrometric system of claim 9, wherein the vibrometer is configured to be secured at a desired angular position with respect to a rotational axis thereof

12. The vibrometric system of claim 9, wherein the probe has a 1500 μm range of displacement.

13. The vibrometric system of claim 9, wherein the probe oscillations follow a substantially sine wave function.

14. The vibrometric system of claim 9, wherein the probe oscillations follow a substantially triangle wave function.

15. The vibrometric system of claim 9, further comprising:
a surround at least partially surrounding the probe; and
a force sensor configured to measure the force applied to the surround.

16. A method of determining existence of peripheral neuropathy in/on a patient's skin, the method comprising:
presenting a test subject with a first time period during which vibrotactile stimulation is applied to a test subject's skin to stimulate a desired group of mechanoreceptors;
receiving a first feedback from the test subject related to the test subject's sensation during the first time period;
presenting the test subject with a second time period during which vibrotactile stimulation is not applied to the test subject's skin;
receiving a second feedback from the test subject related to the test subject's sensation during the second time period; and
comparing the second feedback received from the test subject related to the test subject's sensation during the second time period and the first feedback received from the test subject related to the test subject's sensation during the first time period to design values.

17. The method of claim 16, wherein the vibrotactile stimulation comprises:
placing test subject's skin in contact with a probe of a vibrometer; and
moving the probe of the vibrometer at a desired frequency between 1 and 500 Hz.

18. The method of claim 16, further comprising:
presenting a test subject with a third time period during which vibrotactile stimulation is applied to a test subject's skin to stimulate a desired group of mechanoreceptors;
receiving a third feedback from the test subject related to the test subject's sensation during the third time period;
presenting the test subject with a fourth time period during which vibrotactile stimulation is not applied to the test subject's skin;
receiving a fourth feedback from the test subject related to the test subject's sensation during the fourth time period, wherein the third time period and the fourth time period are presented at random; and
comparing the fourth feedback received from the test subject related to the test subject's sensation during the fourth time period and the third feedback received from the test subject related to the test subject's sensation during the third time period to design values.

19. The method of claim 16, further comprising increasing amplitude of the vibrotactile stimulation if one or more of the test subject feedbacks are different from the design values.

20. The method of claim 16, further comprising decreasing the amplitude of the vibrotactile stimulation if one or more of the test subject's feedbacks match the design values.
